(19) United States
(12) Patent Application Publication
(10) Pub. No.: US 2025/0302424 A1
(43) Pub. Date: Oct. 2, 2025

(54) AUSCULTATION DEVICE AND AUSCULTATION SYSTEM

(71) Applicant: Murata Manufacturing Co., Ltd., Kyoto (JP)

(72) Inventors: Hirofumi Tsuchimoto, Kyoto (JP); Koji Tanaka, Kyoto (JP); Shingo Yamada, Kyoto (JP)

(21) Appl. No.: 19/084,832

(22) Filed: Mar. 20, 2025

(30) Foreign Application Priority Data
Mar. 29, 2024 (JP) 2024-057137

(51) Int. Cl. A61B 7/04

(52) U.S. Cl. CPC A61B 7/04

(57) ABSTRACT

An auscultation device includes a diaphragm in contact with a living body, a sound sensor that receives a vibration propagated from the diaphragm and converts the vibration into an electric signal, and a vibration suppression member that suppresses vibration. The diaphragm includes a first vibration region having a first natural frequency and a second vibration region having a second natural frequency, and when viewed in a first direction in which the diaphragm is in contact with the living body, the first and second vibration regions Ra and Rb are arranged next to each other in a second direction intersecting the first direction. The vibration suppression point is located between the first vibration region and the second vibration region when viewed in the first direction and is offset in the second direction from a center point of the diaphragm in the second direction when viewed in the first direction.

### CROSS REFERENCE TO RELATED APPLICATION

This application claims priority from Japanese Patent Application No. 2024-057137 filed on Mar. 29, 2024. The content of this application is incorporated herein by reference in its entirety.

### BACKGROUND

The present disclosure relates to an auscultation device and an auscultation system.

For example, Japanese Unexamined Patent Application Publication No. 2023-16317 discloses an electronic stethoscope (auscultation device) equipped with multiple sensors for capturing biological sounds. The multiple (four) sensors are arranged in a parallelogram shape when viewed in a contact direction in which the electronic stethoscope is in contact with a living body. Based on magnitudes of biological sounds (vibrations) captured by the respective multiple sensors, a direction in which a sound source producing biological sounds (e.g., a heart producing heart sounds) is located is identified. Identifying the direction in which the sound source is located enables the electronic stethoscope to be brought into contact with a portion of a living body near the sound source, and thus the biological sounds produced from the sound source can be captured more clearly.

However, the electronic stethoscope described in Japanese Unexamined Patent Application Publication No. 2023-16317 is large and complicated because this electronic stethoscope is equipped with the multiple sensors that are brought into contact with the living body and capture the biological sounds.

### BRIEF SUMMARY

The present disclosure achieves a compact and simple structure in an auscultation device capable of identifying a direction in which a sound source that produces biological sounds is located.

In order to solve the above technical problems, according to an aspect of the present disclosure, there is provided an auscultation device including a diaphragm having a first face in contact with a living body and a second face opposite to the first face, a sound sensor configured to receive a vibration propagated from the diaphragm and convert the vibration into an electric signal, a housing configured to support the diaphragm and house the sound sensor, and a vibration suppression member configured to suppress vibration at a vibration suppression point on the diaphragm, in which the diaphragm includes a first vibration region having a first natural frequency and a second vibration region having a second natural frequency different from the first natural frequency, when viewed in a first direction in which the diaphragm is in contact with the living body, the first and second vibration regions are arranged next to each other in a second direction intersecting the first direction, and the vibration suppression point is located between the first vibration region and the second vibration region when viewed in the first direction, and is offset in the second direction from a center point of the diaphragm in the second direction when viewed in the first direction.

According to another aspect of the present disclosure, there is provided an auscultation device including a diaphragm having a first face in contact with a living body and a second face opposite to the first face, a sound sensor configured to receive a vibration propagated from the diaphragm and convert the vibration into an electric signal, and a housing configured to support the diaphragm and house the sound sensor, in which the diaphragm includes a first vibration region having a first natural frequency and a second vibration region having a second natural frequency different from the first natural frequency, when viewed in a first direction in which the diaphragm is in contact with the living body, the first and second vibration regions are arranged next to each other in a second direction intersecting the first direction, and the first and second vibration regions differ in at least one of Young's modulus, density, and thickness, to have the first and second natural frequencies different from each other.

According to another aspect of the present disclosure, there is provided an auscultation system including the auscultation device described above, a processor, and a notification device, in which the processor is configured to process the electric signal from the sound sensor of the auscultation device, detect peak values of the first and second natural frequencies, and identify, based on a magnitude relationship between the peak values of the first and second natural frequencies, whether a sound source in the living body is located on one side or another side in a direction in which the first and second vibration regions are arranged next to each other, and the notification device notifies a direction which is identified by the processor and in which the sound source is located.

According to the present disclosure, a compact and simple structure can be achieved in an auscultation device capable of identifying a direction in which a sound source that produces biological sounds is located.

### BRIEF DESCRIPTION OF THE DRAWINGS

FIG. 1 is a schematic perspective view of an auscultation device according to Embodiment 1;

FIG. 2 is a sectional view of the auscultation device taken along line A-A in FIG. 1;

FIG. 3 is an exploded perspective view of the auscultation device;

FIG. 4 is a top view of the auscultation device illustrating multiple vibration regions of a diaphragm;

FIG. 5 is a conceptual diagram illustrating a vibration system of the diaphragm;

FIG. 6 is a diagram illustrating an example of a frequency spectrum of vibration of the diaphragm of the auscultation device;

FIG. 7 is a block diagram of an example of an auscultation system including the auscultation device;

FIG. 8 is a bottom view of the auscultation device including multiple indicators that indicate directions in which a sound source is located;

FIG. 9 is a sectional view of an auscultation device according to Embodiment 2;

FIG. 10 is a sectional view of an auscultation device according to Embodiment 3;

FIG. 11 is a top view of an auscultation device according to Embodiment 4;

FIG. 12 is a top view of an auscultation device according to Embodiment 5;

FIG. 13 is a diagram for describing a method of identifying, using vectors, a direction in which a sound source is located;

FIG. 14 is a top view of an auscultation device according to Embodiment 6; and

FIG. 15 is a top view of an auscultation device according to another embodiment.

### DETAILED DESCRIPTION

Hereinafter, embodiments of the present disclosure will be described with reference to the drawings.

### Embodiment 1

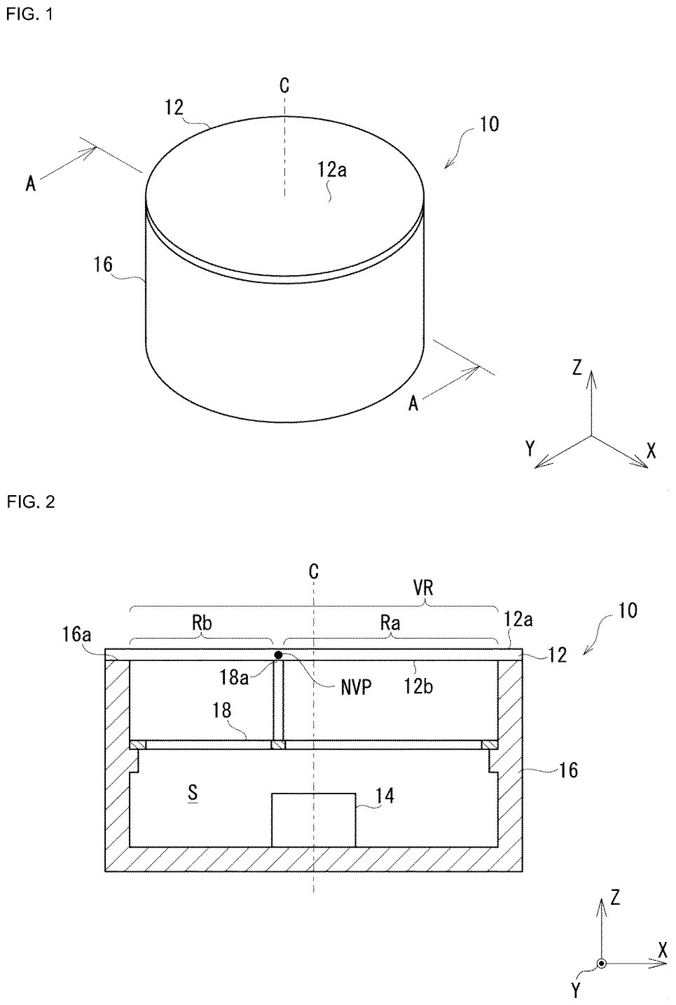

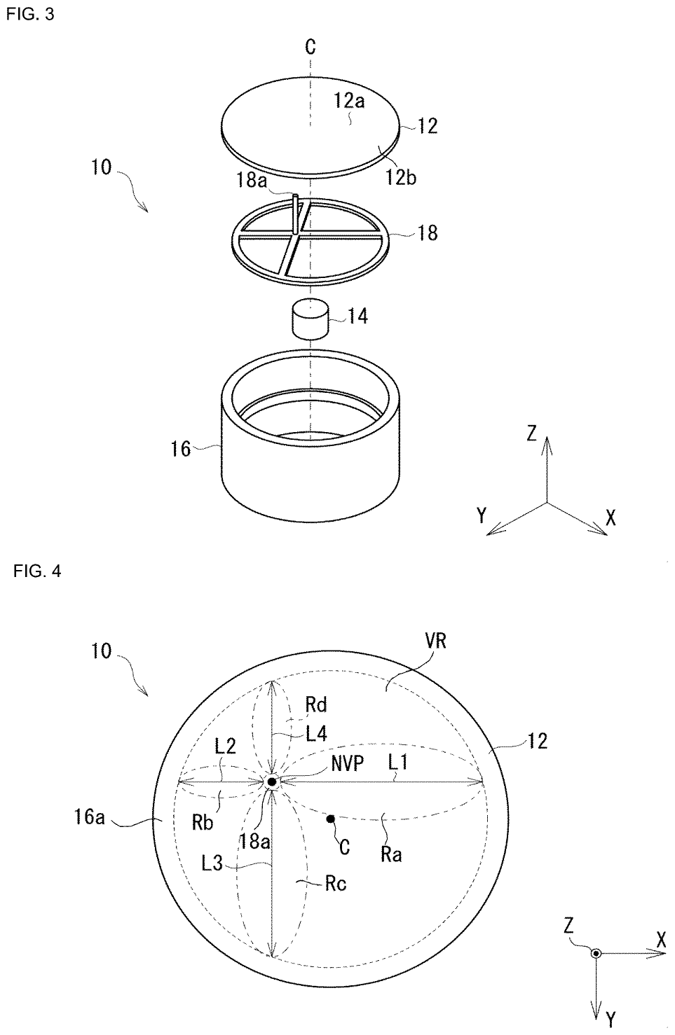

FIG. 1 is a schematic perspective view of an auscultation device according to Embodiment 1 of the present disclosure. FIG. 2 is a sectional view of the auscultation device according to Embodiment 1 taken along line A-A illustrated in FIG. 1. FIG. 3 is an exploded perspective view of the auscultation device according to Embodiment 1. Note that an X-Y-Z orthogonal coordinate system illustrated in the drawings is intended to facilitate understanding of the embodiments of the present disclosure and is not intended to limit the embodiments. Note that an X-axis direction indicates a width direction of the auscultation device, a Y-axis direction indicates a depth direction thereof, and a Z-axis direction (first direction) indicates a thickness direction thereof. The Z-axis direction is a direction in which the auscultation device is in contact with a living body.

An auscultation device 10 according to Embodiment 1 illustrated in FIG. 1 is an electronic device that collects biological sounds produced by a living body, such as a person, in contact with the living body. As illustrated in FIGS. 1 to 3, the auscultation device 10 includes a diaphragm 12 that is in contact with a living body, and a sound sensor 14 that receives vibrations propagated from the diaphragm 12 and converts the vibrations into electric signals. The auscultation device 10 also includes a housing 16 that supports the diaphragm 12 and houses the sound sensor 14.

The diaphragm 12 is a flexible sheet-shaped member made of, for example, an epoxy resin. In Embodiment 1, the diaphragm 12 has a circular shape when viewed in the thickness direction thereof (Z-axis direction). The diaphragm 12 has a first face 12a that is in contact with a living body and a second face 12b opposite to the first face 12a. When the diaphragm 12 is in contact with a living body, the diaphragm 12 vibrates at a frequency and an amplitude corresponding to biological sounds (e.g., heart sounds) produced by the living body.

The sound sensor 14 is located in the housing 16, receives vibrations (i.e., biological sounds) propagated from the diaphragm 12 into the housing 16, and converts the vibrations into electric signals (biological sound data). The sound sensor 14 is, for example, a microphone.

Note that the sound sensor 14 may convert biological sounds into electric signals using an electrodynamic method, an electrostatic method, a piezoelectric method, or the like. In the embodiments of the present disclosure, the method of converting the biological sounds into the electric signals is not limited.

The housing 16 is a so-called chestpiece, which is a part of the auscultation device 10 that is held by a user, such as a doctor, when in use. The housing 16 is made of a rigid material and is cylindrical in Embodiment 1. The housing 16 has an end face 16a to which the diaphragm 12 is attached. The diaphragm 12 is fixed to the end face 16a of the housing 16 with, for example, an adhesive, a double-sided tape, or the like interposed therebetween.

The housing 16 is provided with an internal space S extending in the thickness direction (Z-axis direction). The internal space S is open at the end face 16a to which the diaphragm 12 is attached. Thus, the second face 12b of the diaphragm 12, which is fixed to the end face 16a, faces the internal space S. As a result, the vibrations of the diaphragm 12 propagate to the internal space S.

The sound sensor 14 is housed in the internal space S in the housing 16. Thus, the vibrations from the diaphragm 12 propagate to the sound sensor 14 via the internal space S in the housing 16. As a result, the sound sensor 14 can collect the biological sounds from the living body in contact with the first face 12a of the diaphragm 12 via the diaphragm 12 and the internal space S in the housing 16.

The auscultation device 10 is configured to identify a location of a sound source (e.g., a heart) in a living body in contact with the first face 12a of the diaphragm 12. To be specific, the auscultation device 10 is configured to identify a direction in which the sound source is located relative to the auscultation device 10 when viewed in a direction in which the diaphragm 12 of the auscultation device 10 is in contact with the living body (Z-axis direction).

In order to identify the direction in which the sound source is located relative to the auscultation device 10, the diaphragm 12 includes multiple vibration regions with different natural frequencies.

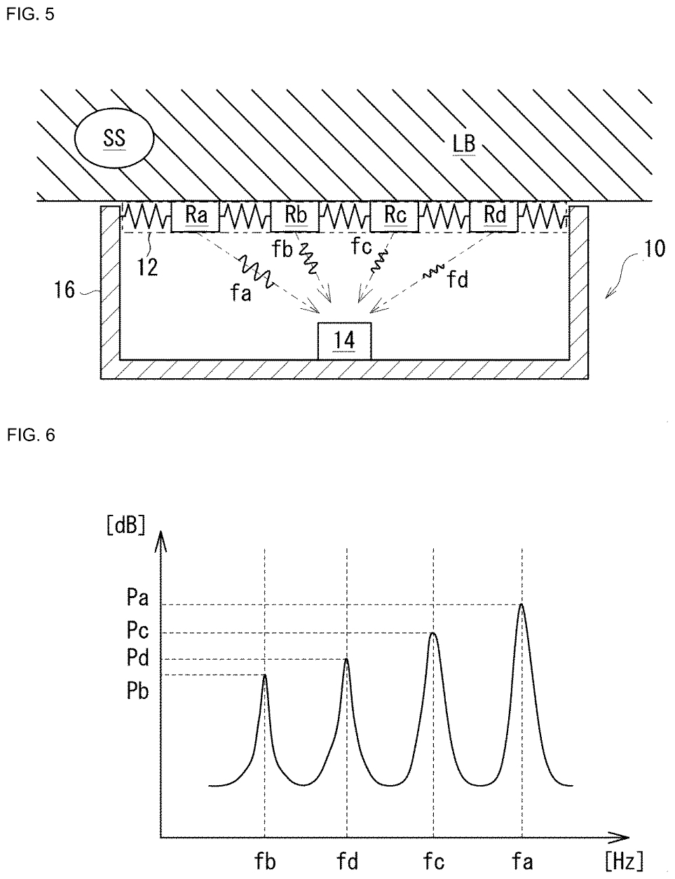

FIG. 4 is a top view of the auscultation device illustrating the multiple vibration regions of the diaphragm. FIG. 5 is a conceptual diagram illustrating a vibration system of the diaphragm. FIG. 6 is a diagram illustrating an example of a frequency spectrum of vibration of the diaphragm of the auscultation device.

As illustrated in FIG. 4, the diaphragm 12 includes multiple regions, namely, first to fourth vibration regions Ra, Rb, Rc, and Rd with different natural frequencies from one another. Note that the first to fourth vibration regions Ra to Rd are located in a portion not fixed to the end face 16a of the housing 16, that is, located in a region that is able to be vibrated (vibration-enabled region) VR facing the internal space S in the housing 16.

In Embodiment 1, the diaphragm 12 has a uniform thickness and is made of a single material. Therefore, the diaphragm 12 cannot include the first to fourth vibration regions Ra to Rd with different natural frequencies as it is. As illustrated in FIGS. 2 and 3, the auscultation device 10 has a vibration suppression member 18 that locally limits the vibration of the diaphragm 12 so that the diaphragm 12 includes the first to fourth vibration regions Ra to Rd with different natural frequencies.

The vibration suppression member 18 has a tip 18a that is in contact with and fixed to the second face 12b of the diaphragm 12 so that the tip 18a overlaps a vibration suppression point NVP on the diaphragm 12 when viewed in the direction in which the auscultation device 10 is in contact with the living body (Z-axis direction). That is, the vibration suppression member 18 is housed in the housing 16. The vibration suppression member 18 suppresses displacement of the vibration suppression point NVP on the diaphragm 12 in the thickness direction of the diaphragm 12 (Z-axis direction).

As illustrated in FIG. 4, the vibration suppression point NVP at which the vibration is limited by the vibration suppression member 18 is offset from the center C of the vibration-enabled region VR of the diaphragm 12 (i.e., a central axis C of the cylindrical auscultation device 10) when viewed in the direction in which the auscultation device 10 is in contact with the living body (Z-axis direction). To be specific, the vibration suppression point NVP is offset from the center C of the vibration-enabled region VR so that distances L1, L2, L3, and L4 in four directions that differ by 90 degrees each from the vibration suppression point NVP to a peripheral end of the vibration-enabled region VR are different from one another.

Providing such a vibration suppression point NVP enables the four vibration regions Ra to Rd with different natural frequencies to be formed. To be specific, when viewed in the direction in which the auscultation device 10 is in contact with the living body (Z-axis direction), the first vibration region Ra and the second vibration region Rb are formed next to each other in the width direction of the auscultation device 10 (X-axis direction) with the vibration suppression point NVP interposed therebetween, and the third vibration region Rc and the fourth vibration region Rd are formed next to each other in the depth direction (Y-axis direction) with the vibration suppression point NVP interposed therebetween. That is, the first to fourth vibration regions Ra to Rd are arranged next to each other in a circumferential direction of the diaphragm 12.

As illustrated in FIG. 5, when the diaphragm 12 is maintained in contact with a living body LB, the entire diaphragm 12 is excited by biological sounds (i.e., vibrations) from a sound source SS (e.g., a heart) in the living body LB. When the entire diaphragm 12 is excited, the first to fourth vibration regions Ra to Rd vibrate at natural frequencies fa, fb, fc, and fd, respectively. Thus, vibrations of the frequency fa propagate from the first vibration region Ra to the sound sensor 14. Similarly, vibrations of the frequency fb propagate from the second vibration region Rb to the sound sensor 14, vibrations of the frequency fc propagate from the third vibration region Rc to the sound sensor 14, and vibrations of the frequency fd propagate from the fourth vibration region Rd to the sound sensor 14.

Note that portions of the diaphragm 12 in the vibration-enabled region VR other than the first to fourth vibration regions Ra to Rd also vibrate, and vibrations propagate to the sound sensor 14.

The sound sensor 14 receives vibrations (i.e., biological sounds) in which vibrations of various frequencies from the diaphragm 12 are superimposed. The sound sensor 14 converts the vibrations into electric signals and outputs the electric signals.

When fast Fourier transform (FFT) processing is performed on the electric signals output from the sound sensor 14, a frequency spectrum indicating intensities of various frequencies contained in a vibration waveform from the diaphragm 12 can be obtained, as illustrated in FIG. 6. From this frequency spectrum, peak values Pa, Pb, Pc, and Pd of the natural frequencies fa to fd of the first to fourth vibration regions Ra to Rd can be obtained.

Magnitudes of the respective peak values Pa to Pd of the natural frequencies fa to fd in the frequency spectrum correspond to distances from the sound source SS to the first to fourth vibration regions Ra to Rd. That is, the vibration region having the natural frequency with the largest peak value is closest to the sound source, and the vibration region having the natural frequency with the smallest peak value is farthest from the sound source. In the example illustrated in FIG. 6, the peak value Pa of the natural frequency fa is the largest, and the peak value Pb of the natural frequency fb is the smallest. In this case, the sound source SS is located on the first vibration region Ra side in a direction in which the first and second vibration regions Ra and Rb are arranged next to each other (X-axis direction).

Note that, in addition, even when the sound source SS is located directly below the center of the sound sensor 14, the peak values Pa, Pb, Pc, and Pd may be non-uniform values due to, for example, directivity or frequency dependency of the sound sensor 14. In this case, peak values when the sound source SS is located directly below the center of the sensor are set as correction values (Pa0, Pb0, Pc0, and Pd0), and a magnitude relationship between relative peak values calculated by subtracting the correction values from the actually measured peak values Pa, Pb, Pc, and Pd is used to identify the position of the sound source.

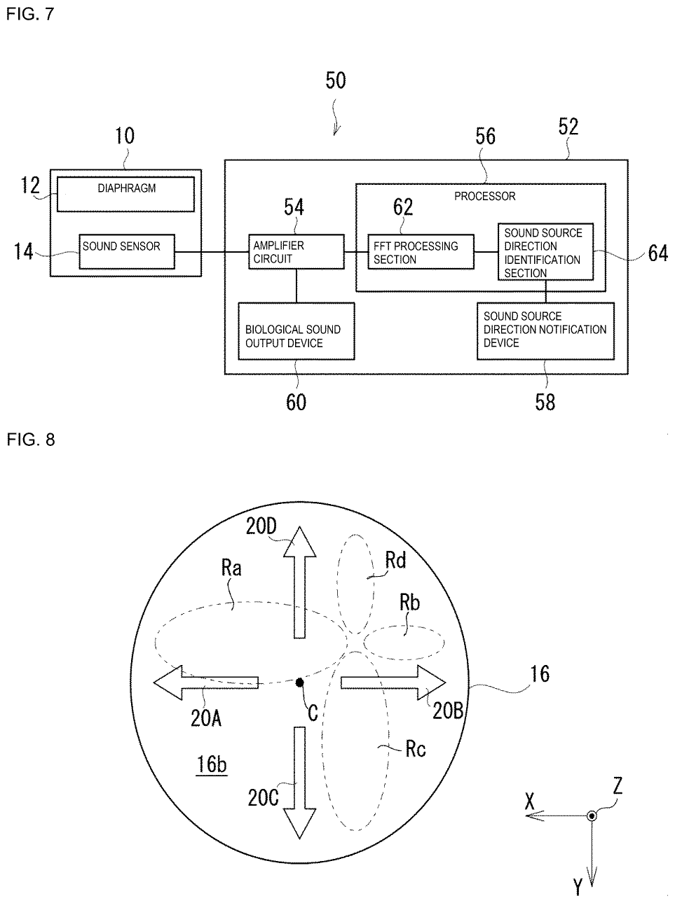

FIG. 7 is a block diagram of an example of an auscultation system including the auscultation device.

An auscultation system 50 illustrated in FIG. 7 is composed of the auscultation device 10 and a mobile terminal 52. The mobile terminal 52 identifies the direction in which the sound source is located relative to the auscultation device 10 and notifies the user of the direction in which the identified sound source is located.

To be specific, the mobile terminal 52 includes an amplifier circuit 54 that amplifies electric signals output from the sound sensor 14 of the auscultation device 10, a processor 56, such as a CPU, that identifies, based on the electric signals output from the amplifier circuit 54, the direction in which the sound source is located relative to the auscultation device 10, and a sound source direction notification device 58 that notifies the direction in which the sound source identified by the processor 56 is located. The mobile terminal 52 also includes a biological sound output device 60 that converts the electric signals output from the amplifier circuit 54 into biological sound data and outputs the biological sound data.

The auscultation device 10 and the mobile terminal 52 are connected via a wire or wirelessly. When wirelessly connected, the auscultation device 10 and the mobile terminal 52 are each equipped with a wireless communication device. Thus, the electric signals (biological sound data) output from the sound sensor 14 of the auscultation device 10 are transmitted to the mobile terminal 52.

The amplifier circuit 54 of the mobile terminal 52 amplifies the electric signals output from the auscultation device 10. The amplified electric signals are output to the processor 56 and the biological sound output device 60.

The processor 56 includes an FFT processing section 62 that performs FFT processing on the electric signals output from the amplifier circuit 54, and a sound source direction identification section 64 that identifies the direction in which the sound source is located relative to the auscultation device 10, based on the frequency spectrum obtained by the FFT processing performed by the FFT processing section 62. For example, the processor 56 functions as the FFT processing section 62 and the sound source direction identification section 64 by operating in accordance with a program stored in a storage device (not illustrated) such as a memory or a hard disk.

The FFT processing section 62 of the processor 56 performs FFT processing on the electric signals output from the amplifier circuit 54, that is, the vibration waveform propagated from the diaphragm 12 and received by the sound sensor 14, and creates frequency spectrum data indicating the intensities of various frequencies contained in the vibration waveform from the diaphragm 12.

The sound source direction identification section 64 of the processor 56 first detects, based on the frequency spectrum data created by the FFT processing section 62, the peak values Pa to Pd of the respective natural frequencies fa to fd of the first to fourth vibration regions Ra to Rd. Subsequently, the sound source direction identification section 64 calculates the magnitude relationship between the peak values Pa to Pd of the natural frequencies fa to fd by comparing the detected peak values Pa to Pd. Based on the magnitude relationship between the calculated peak values Pa to Pd and a positional relationship between the first to fourth vibration regions Ra to Rd, the sound source direction identification section 64 identifies the direction in which the sound source SS is located relative to the auscultation device 10.

The sound source direction notification device 58 notifies the user of the auscultation device 10 of the direction in which the sound source SS is located relative to the auscultation device 10 and which has been identified by the sound source direction identification section 64 of the processor 56.

FIG. 8 is a bottom view of the auscultation device including multiple indicators that indicate directions in which a sound source is located.

In order to indicate to the user the direction in which the sound source SS is located relative to the auscultation device 10, the auscultation device 10 includes multiple arrow-shaped indicators 20A, 20B, 20C, and 20D, which indicate directions that differ by 90 degrees each, on a bottom face 16b of the housing 16 (an end face opposite to the end face 16a that supports the diaphragm 12). The indicators 20A and 20B indicate the width direction of the auscultation device 10 (X-axis direction) and point in opposite directions to each other. The indicators 20C and 20D indicate the depth direction of the auscultation device 10 (Y-axis direction) and point in opposite directions to each other. The indicators 20A to 20D are light-emitting devices, such as LEDs. Using the four arrow-shaped indicators 20A to 20D can notify the user of the direction in which the sound source SS is located.

To be specific, the sound source direction notification device 58 turns on the indicator corresponding to the vibration region having the natural frequency with the largest peak value. For example, when the peak value Pa of the natural frequency fa is the largest, the sound source direction notification device 58 causes the indicator 20A corresponding to the first vibration region Ra to emit light. Thus, the user can know that the sound source SS is located in the direction indicated by the indicator 20A, and can move the auscultation device 10 closer to the sound source SS.

Note that when the peak values Pa to Pd of the natural frequencies fa to fd are substantially the same, the sound source SS is located at a position facing the first face 12a of the diaphragm 12. In this case, all the indicators 20A to 20D are turned on. Thus, the user can know that the sound source SS is located under the auscultation device 10.

Notification of the direction in which the sound source is located by the sound source direction notification device 58 is not limited to notification by the indicators 20A to 20D. For example, when the mobile terminal 52 includes a display, the sound source direction notification device 58 may display on the display the auscultation device 10 and an arrow indicating the direction in which the sound source is located. Alternatively, the sound source direction notification device 58 may notify, by voice, the direction in which the sound source is located.

The biological sound output device 60 of the mobile terminal 52 converts the electric signals output from the amplifier circuit 54 into biological sound data and stores the biological sound data in a storage device (not illustrated) of the mobile terminal 52. The biological sound output device 60 displays the biological sound data on the display of the mobile terminal 52.

Note that the auscultation device 10 may have some or all of the functions of the mobile terminal 52. For example, the amplifier circuit 54, the processor 56, and the sound source direction notification device 58 may be mounted on the auscultation device 10. In this case, the mobile terminal 52 includes the biological sound output device 60.

According to Embodiment 1 as described above, a compact and simple structure can be achieved in an auscultation device capable of identifying a direction in which a sound source that produces biological sounds is located.

### Embodiment 2

Embodiment 2 is an improvement of Embodiment 1 described above and differs from Embodiment 1 in a method of forming multiple vibration regions with different natural frequencies in a diaphragm. Therefore, Embodiment 2 will be described with a focus on the difference. Note that the same symbols are given to components that are substantially the same as the components of the above-described embodiment.

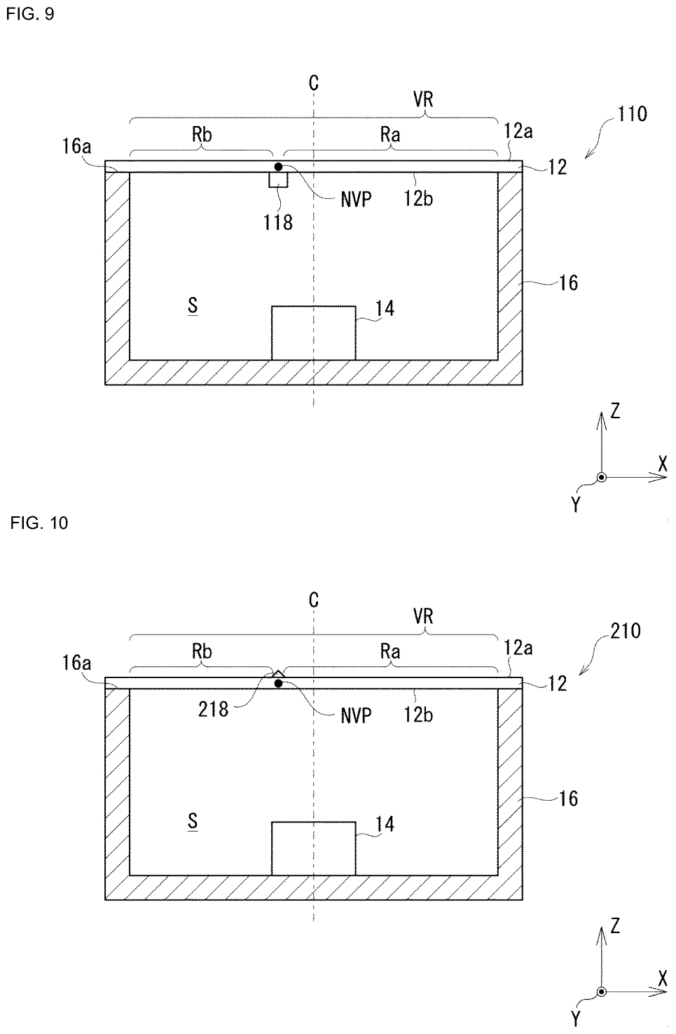

FIG. 9 is a sectional view of an auscultation device according to Embodiment 2.

As illustrated in FIG. 9, in an auscultation device 110 according to Embodiment 2, a diaphragm 12 has a uniform thickness and is made of a single material. A weight 118 as a vibration suppression member is located on a second face 12b of the diaphragm 12 so as to overlap a vibration suppression point NVP on the diaphragm 12 when viewed in a direction in which the auscultation device 110 is in contact with a living body (Z-axis direction). The weight 118 suppresses displacement of the vibration suppression point NVP compared to displacement of other portions of the diaphragm 12 in a vibration-enabled region VR.

In Embodiment 2, as in Embodiment 1 described above, the vibration suppression point NVP is offset from the center C of the vibration-enabled region VR of the diaphragm 12 (i.e., a central axis C of the cylindrical auscultation device 110) when viewed in the direction in which the auscultation device 110 is in contact with the living body (Z-axis direction). To be specific, the vibration suppression point NVP is offset from the center C of the vibration-enabled region VR so that distances in four directions that differ by 90 degrees each from the vibration suppression point NVP to a peripheral end of the vibration-enabled region VR are different from one another.

As a result, the diaphragm 12 in the auscultation device 110 according to Embodiment 2 can include multiple vibration regions with different natural frequencies, as in Embodiment 1 described above.

As in Embodiment 1 described above, in Embodiment 2 described above, a compact and simple structure can be achieved in an auscultation device capable of identifying a direction in which a sound source that produces biological sounds is located.

### Embodiment 3

Embodiment 3 is an improvement of Embodiment 1 described above and differs from Embodiment 1 in a method of forming multiple vibration regions with different natural frequencies in a diaphragm. Therefore, Embodiment 3 will be described with a focus on the difference. Note that the same symbols are given to components that are substantially the same as the components of the above-described embodiments.

FIG. 10 is a sectional view of an auscultation device according to Embodiment 3.

As illustrated in FIG. 10, in an auscultation device 210 according to Embodiment 3, a diaphragm 12 has a uniform thickness and is made of a single material. A projection 218 as a vibration suppression member is provided on a first face 12a of the diaphragm 12 so as to overlap a vibration suppression point NVP on the diaphragm 12 when viewed in a direction in which the auscultation device 210 is in contact with a living body (Z-axis direction). The projection 218 is pressed onto skin of a living body in contact with the first face 12a of the diaphragm 12, thereby suppressing displacement of the vibration suppression point NVP compared to displacement of other portions of the diaphragm 12 in a vibration-enabled region VR.

In Embodiment 3, as in Embodiment 1 described above, the vibration suppression point NVP is offset from the center of the vibration-enabled region VR of the diaphragm 12 (i.e., a central axis C of the cylindrical auscultation device 210) when viewed in the direction in which the auscultation device 210 is in contact with the living body (Z-axis direction). To be specific, the vibration suppression point NVP is offset from the center C of the vibration-enabled region VR so that distances in four directions that differ by 90 degrees each from the vibration suppression point NVP to a peripheral end of the vibration-enabled region VR are different from one another.

As a result, as in Embodiment 1 described above, the diaphragm 12 in the auscultation device 210 according to Embodiment 3 can include multiple vibration regions with different natural frequencies, as in Embodiment 1 described above.

As in Embodiment 1 described above, in Embodiment 3 described above, a compact and simple structure can be achieved in an auscultation device capable of identifying a direction in which a sound source that produces biological sounds is located.

### Embodiment 4

In Embodiments 1 to 3 described above, the diaphragm 12 in the auscultation devices 10, 110, and 210 has a uniform thickness and is made of a single material. In order for such a diaphragm 12 to include multiple vibration regions with different natural frequencies, the auscultation devices 10, 110, and 210 include the vibration suppression members 18, 118, and 218 that locally limit the vibration of the diaphragm, respectively. In contrast, a diaphragm in an auscultation device according to Embodiment 4 includes multiple vibration regions with different natural frequencies without necessarily using any other members. Therefore, Embodiment 4 will be described with a focus on this different diaphragm. Note that the same symbols are given to components that are substantially the same as the components of the above-described embodiments.

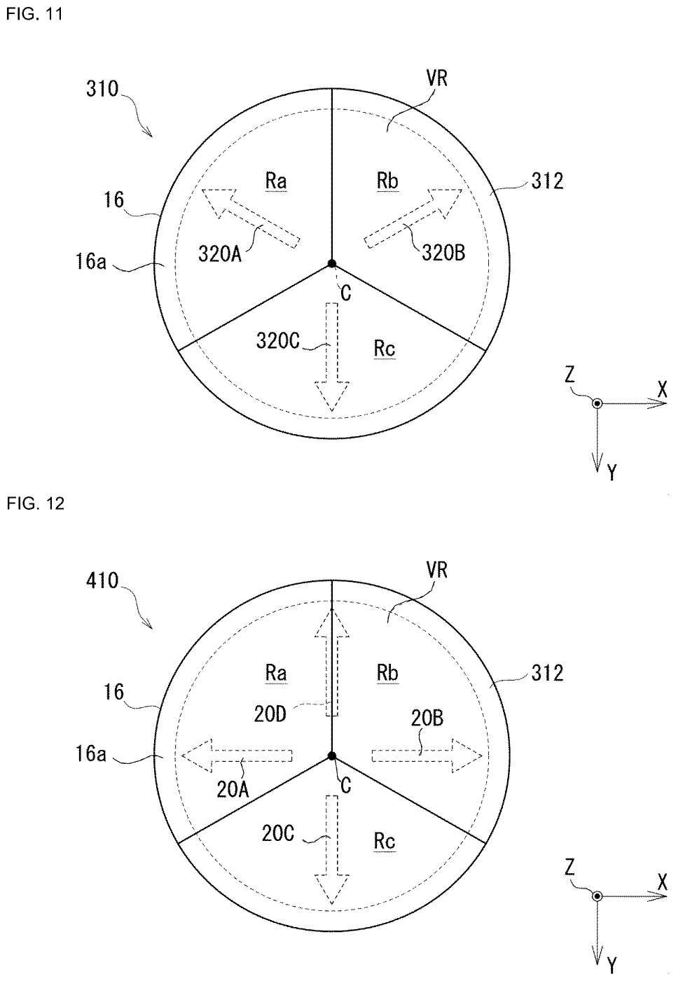

FIG. 11 is a top view of the auscultation device according to Embodiment 4.

As illustrated in FIG. 11, in an auscultation device 310 according to Embodiment 4, a diaphragm 312 includes first to third vibration regions Ra, Rb, and Rc with the same fan shape when viewed in a direction in which the auscultation device 310 is in contact with a living body (Z-axis direction). The first to third vibration regions Ra to Rc are arranged next to each other in a circumferential direction of the diaphragm 312.

The first to third vibration regions Ra to Rc of the diaphragm 312 have different physical properties from one another. Thus, the first to third vibration regions Ra to Rc have different natural frequencies from one another.

To be specific, vibration behavior of each of the first to third vibration regions Ra to Rc is similar to vibration behavior of a double-supported beam. Therefore, the natural frequency of each vibration region can be expressed by Equation 1 below.

$${f}_{n} = \frac{{λ}_{n}^{2}}{2 π} \sqrt{\frac{EI}{ρ A}} \qquad ( Equation 1 )$$

In Equation 1, n is an integer of 1 or more. fn is a natural frequency of a nth mode. E is Young's modulus. I is the second moment of area. ρ is density. A is an average cross-sectional area of the vibration region that intersects a radial direction of the diaphragm. λn is a constant that satisfies Equation 2 below.

$${λ}_{n} = \frac{n π}{L} \qquad ( Equation 2 )$$

In Equation 2, L is a length of the vibration region (i.e., a radius of the diaphragm).

As shown in Equation 1, the natural frequencies of the first to third vibration regions Ra to Rc differ from one another when the Young's moduli or the densities are different, that is, when the materials are different. Further, when the cross-sectional areas are different, that is, when the thicknesses are different, the natural frequencies of the first to third vibration regions Ra to Rc are different from one another. Note that the thicknesses of the first to third vibration regions can be different from one another, for example, by laminating different numbers of fan-shaped sheets made of the same material.

Therefore, the first to third vibration regions Ra to Rc in the diaphragm 312 can have natural frequencies different from one another by being different in at least one of the Young's modulus, the density, and the thickness.

The auscultation device 310 according to Embodiment 4 includes three arrow-shaped indicators 320A, 320B, and 320C that are located on a bottom face of a housing 16 and indicate directions that differ by 120 degrees each. The three arrow-shaped indicators 320A to 320C can be used to notify the direction in which the sound source is located. For example, when a peak value of a natural frequency of the first vibration region Ra is the largest, the indicator 320A is turned on.

As in Embodiment 1 described above, in Embodiment 4 described above, a compact and simple structure can be achieved in an auscultation device capable of identifying a direction in which a sound source that produces biological sounds is located.

### Embodiment 5

Embodiment 5 is an improvement of Embodiment 4 described above. In Embodiment 4 described above, as illustrated in FIG. 11, the direction of the sound source is identified using the three vibration regions Ra to Rc of the diaphragm 312, and the identified direction is notified to the user using the three arrow-shaped indicators 320A to 320C. In other words, the number of vibration regions and the number of indicators are the same. In contrast, in Embodiment 5, the number of vibration regions and the number of indicators are not the same. Therefore, Embodiment 5 will be described with a focus on this difference. Note that the same symbols are given to components that are substantially the same as the components of the above-described embodiments.

FIG. 12 is a top view of an auscultation device according to Embodiment 5.

As illustrated in FIG. 12, in an auscultation device 410 according to Embodiment 5, a diaphragm 312 includes first to third vibration regions Ra, Rb, and Rc with the same fan shape when viewed in a direction in which the auscultation device 410 is in contact with a living body (Z-axis direction). The first to third vibration regions Ra to Rc are arranged next to each other in a circumferential direction of the diaphragm 312.

The first to third vibration regions Ra to Rc of the diaphragm 312 have different physical properties from one another. Thus, the first to third vibration regions Ra to Rc have different natural frequencies from one another.

The auscultation device 410 according to Embodiment 5 includes four arrow-shaped indicators 20A to 20D that are located on a bottom face of the housing 16 and indicate directions that differ by 90 degrees each. The indicators 20A and 20B indicate a width direction of the auscultation device 410 (X-axis direction) and point in opposite directions to each other. The indicators 20C and 20D indicate a depth direction of the auscultation device 410 (Y-axis direction) and point in opposite directions to each other. By using the four arrow-shaped indicators 20A to 20D, the auscultation device 410 notifies a direction in which a sound source is located. The direction to be notified will be described.

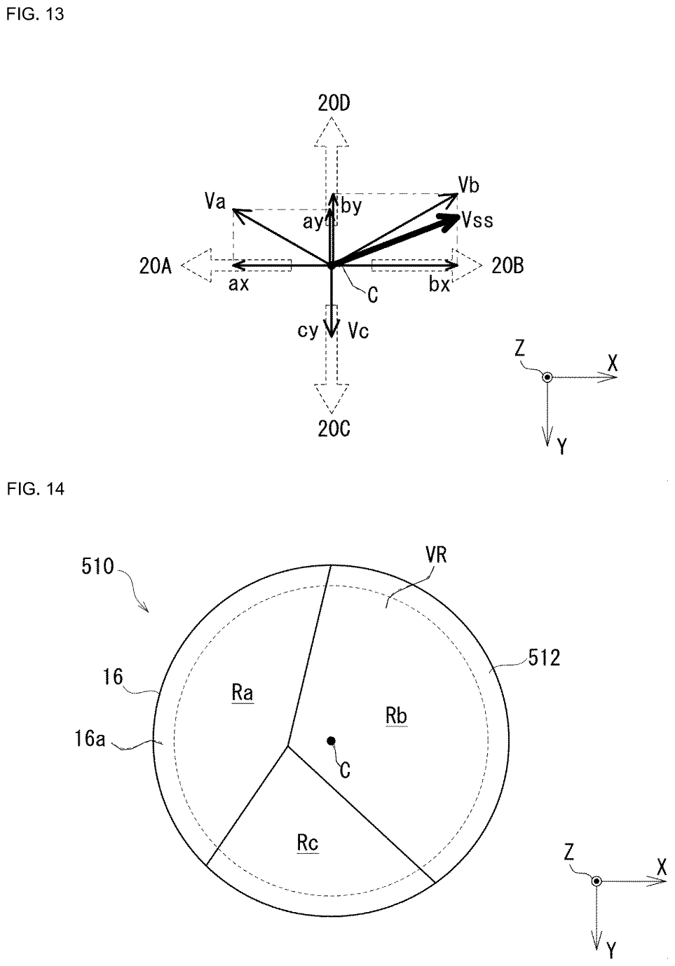

FIG. 13 is a diagram for describing a method of identifying, using vectors, a direction in which a sound source is located.

As illustrated in FIG. 13, in Embodiment 5, peak values of the natural frequencies of the first to third vibration regions Ra to Rc are indicated as vectors. To be specific, for the three fan-shaped first to third vibration regions Ra to Rc, vectors Va, Vb, and Vc are calculated respectively, each of which extends in a direction from a vertex (i.e., the center C of the diaphragm 312) toward the center of an arc-shape outer end and has a magnitude corresponding to the peak value. Subsequently, width direction components ax, bx, and cx (X-axis direction components) and depth components ay, by, and cy (Y-axis direction components) of the vectors Va to Vc are calculated. Note that in the present embodiment, the width direction component cx of the vector Vc is zero. By combining the vector components ax, bx, ay, by, and cy, a vector Vss extending in a direction in which the sound source is located can be calculated. By turning on an indicator that indicates a direction close to the direction indicated by the vector Vss (indicator 20B in the example illustrated in FIG. 13), the auscultation device 10 notifies the user of the direction in which the sound source is located.

As in Embodiment 1 described above, in Embodiment 5 described above, a compact and simple structure can be achieved in an auscultation device capable of identifying a direction in which a sound source that produces biological sounds is located.

### Embodiment 6

In Embodiments 4 and 5 described above, the multiple vibration regions in the diaphragm have the same shape when viewed in the direction in which the auscultation device is in contact with the living body. In Embodiment 6, multiple vibration regions differ from one another in shape. Therefore, Embodiment 6 will be described with a focus on this difference. Note that the same symbols are given to components that are substantially the same as the components of the above-described embodiments.

FIG. 14 is a top view of an auscultation device according to Embodiment 6.

As illustrated in FIG. 14, in an auscultation device 510 according to Embodiment 6, a diaphragm 512 includes first to third vibration regions Ra, Rb, and Rc with different shapes when viewed in a direction in which the auscultation device 510 is in contact with a living body (Z-axis direction). The first to third vibration regions Ra to Rc are arranged next to each other in a circumferential direction of the diaphragm 512.

The first to third vibration regions Ra to Rc of the diaphragm 512 have different physical properties from one another. Thus, the first to third vibration regions Ra to Rc have different natural frequencies from one another.

As in Embodiment 1 described above, in Embodiment 6 described above, a compact and simple structure can be achieved in an auscultation device capable of identifying a direction in which a sound source that produces biological sounds is located.

Although the present disclosure has been described with reference to the multiple embodiments, the embodiments of the present disclosure are not limited thereto.

For example, in Embodiments 4 to 6 described above, the diaphragm has three vibration regions with different natural frequencies. However, the embodiments of the present disclosure are not limited to this. That is, the present disclosure does not limit the number of vibration regions with different natural frequencies on a diaphragm. The number of vibration regions with different natural frequencies on the diaphragm may be, for example, five. That is, when multiple vibration regions with different natural frequencies are arranged next to each other in a circumferential direction of the diaphragm, the direction in which the sound source is located relative to the auscultation device can be identified by comparing the peak values of the natural frequencies of the respective vibration regions.

For reference, an example of a layout of multiple vibration regions in which the direction in which the sound source is located relative to the auscultation device cannot be identified is a layout in which multiple vibration regions are located in concentric circles on a diaphragm.

From another point of view, in Embodiments 1 to 3 described above, various portions of the diaphragm in each vibration-enabled region vibrate at respective natural frequencies thereof. In the multiple portions in the vibration-enabled region, multiple peripheral side portions that are located in different directions relative to the central portion of the diaphragm are used as multiple vibration regions for identifying the direction in which the sound source is located.

In Embodiments 4 to 6 described above, the multiple vibration regions on the diaphragm have natural frequencies different from one another. That is, the natural frequency greatly changes across a boundary between the vibration regions. However, the embodiments of the present disclosure are not limited to this. The diaphragm may have multiple vibration regions so that the natural frequency changes continuously. To achieve this, for example, the diaphragm may have a thickness that changes continuously rather than intermittently.

In Embodiments 1 to 6 described above, the diaphragm of the auscultation device includes at least three vibration regions with different natural frequencies. However, the embodiments of the present disclosure are not limited to this.

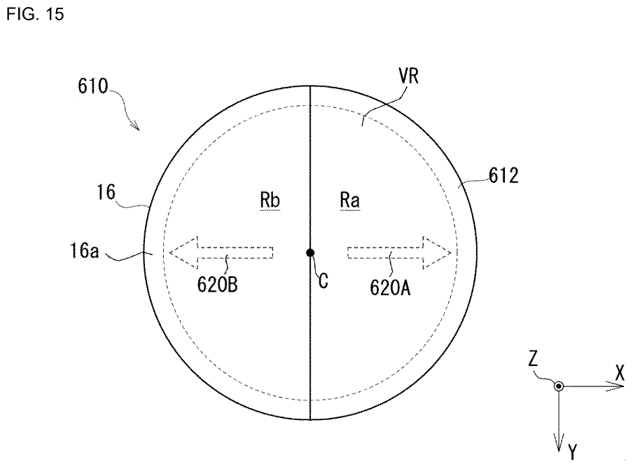

FIG. 15 is a top view of an auscultation device according to another embodiment.

As illustrated in FIG. 15, in an auscultation device 610 according to the other embodiment, a diaphragm 612 includes first and second vibration regions Ra and Rb, which are arranged next to each other in a width direction of the auscultation device 610 (X-axis direction) when viewed in a direction in which the auscultation device 610 is in contact with a living body (Z-axis direction) and have different natural frequencies.

The auscultation device 610 according to the other embodiment includes two arrow-shaped indicators 620A and 620B that are located on a bottom face of a housing 16 and indicate directions that differ by 180 degrees. The indicators 620A and 620B indicate the width direction of the auscultation device 610 (X-axis direction) and point in opposite directions to each other.

In the auscultation device 610 according to the other embodiment, when the sound source is located on one side or another side of the width direction of the auscultation device 610 (X-axis direction), a peak value of the natural frequency of one of the first vibration region Ra and the second vibration region Rb is larger than a peak value of the natural frequency of the other thereof. For example, when the peak value of the natural frequency of the first vibration region Ra is larger than the peak value of the natural frequency of the second vibration region Rb, the indicator 620A is turned on. Thus, the user can know the direction in which the sound source is located.

In contrast, when the sound source is located on one side or another side in a depth direction of the auscultation device 610 (Y-axis direction), the peak values of the natural frequencies of the first and second vibration regions Ra and Rb are substantially equal. In this case, for example, both the indicators 620A and 620B are made to flash to notify the user that the sound source is located on one side or the other side of the depth direction of the auscultation device 610. Based on this notification, the user rotates the auscultation device 610 90 degrees around a central axis C thereof. Thus, the peak value of the natural frequency of one of the first and second vibration regions Ra and Rb is larger than the peak value of the natural frequency of the other thereof. Then, the indicator corresponding to the vibration region having the natural frequency with the larger peak value is turned on. As a result, the user can know the direction in which the sound source is located relative to the auscultation device 610.

In addition, the natural frequency of each of the at least two vibration regions in the diaphragm of the auscultation device can be different from a frequency of the biological sound produced by the sound source in the living body in contact with the auscultation device.

When the frequency produced by the sound source is equal to the natural frequency of the vibration region on the diaphragm, the vibration region may resonate. For example, when a vibration region far from the sound source resonates, accuracy of identifying the direction in which the sound source is located may decrease. In particular, the auscultation device 610 according to the other embodiment illustrated in FIG. 15 may be unable to identify the direction in which the sound source is located or may identify a wrong direction. Therefore, when the living body from which the biological sounds are collected by the auscultation device is identified, that is, when the frequency produced by the sound source in the living body is a predetermined frequency, the natural frequency of each of the multiple vibration regions on the diaphragm can be different from the predetermined frequency.

However, when there are many vibration regions on the diaphragm with different natural frequencies, even if one vibration region resonates, the direction in which the sound source is located can be identified based on the magnitude relationship of the peak values of the natural frequencies of the remaining vibration regions.

That is, various aspects of the present disclosure are as follows.

A first aspect is an auscultation device including a diaphragm having a first face in contact with a living body and a second face opposite to the first face, a sound sensor configured to receive a vibration propagated from the diaphragm and convert the vibration into an electric signal, a housing configured to support the diaphragm and house the sound sensor, and a vibration suppression member configured to suppress vibration at a vibration suppression point on the diaphragm, in which the diaphragm includes a first vibration region having a first natural frequency and a second vibration region having a second natural frequency different from the first natural frequency, when viewed in a first direction in which the diaphragm is in contact with the living body, the first and second vibration regions are arranged next to each other in a second direction intersecting the first direction, and the vibration suppression point is located between the first vibration region and the second vibration region when viewed in the first direction, and is offset in the second direction from a center point of the diaphragm in the second direction when viewed in the first direction.

A second aspect is the auscultation device according to the first aspect, in which the vibration suppression member is a member having a tip in contact with the second face of the diaphragm while the tip overlaps the vibration suppression point of the diaphragm when viewed in the first direction.

A third aspect is the auscultation device according to the first aspect, in which the vibration suppression member is a weight located on the second face of the diaphragm while the weight overlaps the vibration suppression point of the diaphragm when viewed in the first direction.

A fourth aspect is the auscultation device according to the first aspect, in which the vibration suppression member is a projection located on the first face of the diaphragm while the projection overlaps the vibration suppression point of the diaphragm when viewed in the first direction.

A fifth aspect is an auscultation device including a diaphragm having a first face in contact with a living body and a second face opposite to the first face, a sound sensor configured to receive a vibration propagated from the diaphragm and convert the vibration into an electric signal, and a housing configured to support the diaphragm and house the sound sensor, in which the diaphragm includes a first vibration region having a first natural frequency and a second vibration region having a second natural frequency different from the first natural frequency, when viewed in a first direction in which the diaphragm is in contact with the living body, the first and second vibration regions are arranged next to each other in a second direction intersecting the first direction, and the first and second vibration regions differ in at least one of Young's modulus, density, and thickness, to have the first and second natural frequencies different from each other.

A sixth aspect is the auscultation device according to any one of the first to fifth aspects, in which the diaphragm includes a third vibration region having a third natural frequency different from the first and second natural frequencies, and a fourth vibration region having a fourth natural frequency different from the first, second, and third natural frequencies, and when viewed in the first direction, the first, second, third, and fourth vibration regions are arranged next to each other in a circumferential direction of the diaphragm.

A seventh aspect is the auscultation device according to any one of the first to sixth aspects, in which a biological sound produced by the living body in contact with the first face of the diaphragm has a predetermined frequency, and the first and second natural frequencies are different from the predetermined frequency.

A eighth aspect is an auscultation system including the auscultation device according to any one of the first to seventh aspects, a processor, and a notification device, in which the processor is configured to process the electric signal from the sound sensor of the auscultation device, detect peak values of the first and second natural frequencies, and identify, based on a magnitude relationship between the peak values of the first and second natural frequencies, whether a sound source in the living body is located on one side or another side in a direction in which the first and second vibration regions are arranged next to each other, and the notification device notifies a direction which is identified by the processor and in which the sound source is located.

What is claimed is:

1. An auscultation device comprising:
a diaphragm having a first face configured to contact a living body and a second face opposite to the first face;
a sound sensor configured to receive a vibration propagated from the diaphragm and to convert the vibration into an electric signal;
a housing configured to support the diaphragm and house the sound sensor; and
a vibration suppressor configured to suppress vibration at a vibration suppression point on the diaphragm,
wherein the diaphragm includes a first vibration region having a first natural frequency and a second vibration region having a second natural frequency different from the first natural frequency,
wherein when viewed in a first direction in which the diaphragm would be in contact with the living body, the first and second vibration regions are arranged next to each other in a second direction intersecting the first direction, and
wherein the vibration suppression point is located between the first vibration region and the second vibration region when viewed in the first direction, and is offset in the second direction from a center point of the diaphragm in the second direction when viewed in the first direction.

2. The auscultation device according to claim 1, wherein the vibration suppressor has a tip in contact with the second face of the diaphragm while the tip overlaps the vibration suppression point of the diaphragm when viewed in the first direction.

3. The auscultation device according to claim 1, wherein the vibration suppressor is a weight located on the second face of the diaphragm while the weight overlaps the vibration suppression point of the diaphragm when viewed in the first direction.

4. The auscultation device according to claim 1, wherein the vibration suppressor is a projection located on the first face of the diaphragm while the projection overlaps the vibration suppression point of the diaphragm when viewed in the first direction.

5. The auscultation device according to claim 1,
wherein the diaphragm includes a third vibration region having a third natural frequency different from the first and second natural frequencies, and a fourth vibration region having a fourth natural frequency different from the first, second, and third natural frequencies, and
wherein when viewed in the first direction, the first, second, third, and fourth vibration regions are arranged next to each other in a circumferential direction of the diaphragm.

6. The auscultation device according to claim 1,
wherein the first and second natural frequencies are different from a biological sound produced by the living body.

7. An auscultation system comprising:
the auscultation device according to claim 1;
a processor; and
a notification device,
wherein the processor is configured to process the electric signal from the sound sensor of the auscultation device, to detect peak values of the first and second natural frequencies, and to identify, based on a magnitude relationship between the peak values of the first and second natural frequencies, whether a sound source in the living body is located on one side or another side in a direction in which the first and second vibration regions are arranged next to each other, and
wherein the notification device is configured to notify the direction identified by the processor in which the sound source is located.

8. An auscultation device comprising:
a diaphragm having a first face configured to contact a living body and a second face opposite to the first face;
a sound sensor configured to receive a vibration propagated from the diaphragm and to convert the vibration into an electric signal; and
a housing configured to support the diaphragm and house the sound sensor,
wherein the diaphragm includes a first vibration region having a first natural frequency and a second vibration region having a second natural frequency different from the first natural frequency,
wherein when viewed in a first direction in which the diaphragm would be in contact with the living body, the first and second vibration regions are arranged next to each other in a second direction intersecting the first direction, and
wherein the first and second vibration regions have a different Young's modulus, density, or thickness, such that the first and second natural frequencies are different from each other.

9. The auscultation device according to claim 8,
wherein the diaphragm includes a third vibration region having a third natural frequency different from the first and second natural frequencies, and a fourth vibration region having a fourth natural frequency different from the first, second, and third natural frequencies, and
wherein when viewed in the first direction, the first, second, third, and fourth vibration regions are arranged next to each other in a circumferential direction of the diaphragm.

10. The auscultation device according to claim 8,
wherein the first and second natural frequencies are different from a frequency produced by the living body.

11. An auscultation system comprising:
the auscultation device according to claim 8;
a processor; and
a notification device,
wherein the processor is configured to process the electric signal from the sound sensor of the auscultation device, to detect peak values of the first and second natural frequencies, and to identify, based on a magnitude relationship between the peak values of the first and second natural frequencies, whether a sound source in the living body is located on one side or another side in a direction in which the first and second vibration regions are arranged next to each other, and
wherein the notification device is configured to notify the direction identified by the processor in which the sound source is located.
